(19) United States
(12) Patent Application Publication
(10) Pub. No.: US 2008/0276732 A1
(43) Pub. Date: Nov. 13, 2008

(54) INDICATOR FOR ORALLY ADMINISTERED COMPOSITIONS

(75) Inventor: Jason Peters, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: MULTI FORMULATIONS LTD., Mississauga (CA)

(21) Appl. No.: 11/867,450

(22) Filed: Oct. 4, 2007

Related U.S. Application Data
(60) Provisional application No. 60/917,304, filed on May 10, 2007.

(51) Int. Cl. G01N 33/02

(52) U.S. Cl. 73/866

(57) ABSTRACT

A method for estimating the rate of bioavailability of the active components of orally administered dietary supplements of provided. The method comprises the administration of a substance which provides a sensory feedback to a user following administration. For example, Niacin or derivatives thereof provide sate, transient and perceptible sensory effects which are indicative of the bioavailability of active components of the dietary supplemental composition.

### RELATED APPLICATIONS

The application is related to and claims benefit of priority to U.S. Provisional Patent Application Ser. No. 60/917,304 entitled “Indicator for orally administered compositions,” filed May 10, 2007, the disclosure of which is hereby fully incorporated by reference.

### FIELD OF THE INVENTION

The present invention is directed towards a method for identifying the temporal bioavailability of orally administered dietary supplemental compositions. Specifically, a method is provided for indicating and estimating the rate of bioavailability of the active ingredients contained in dietary supplemental compositions for oral administration.

### BACKGROUND OF THE INVENTION

The United States Government defines a ‘dietary supplement’ in the Dietary Supplement Health and Education Act of 1994 as “a product (other than tobacco) that is intended to supplement the diet that bears or contains one or more of the following dietary ingredients: a vitamin, a mineral, a herb or other botanical, an amino acid, a dietary substance for use by man to supplement the diet by increasing the total daily intake, or a concentrate, metabolite, constituent, extract, or combinations of these ingredients” as cited in (Kwan D, Hirschkorn K, Boon H. U.S. and Canadian pharmacists' attitudes, knowledge, and professional practice behaviors toward dietary supplements: a systematic review. BMC Complement Altern Med. 2006 Sep. 19; 6:31).

Nutritional dietary supplements are taken for a number of reasons by a wide array of people. Supplements such as multi-vitamin/mineral complexes for general health and more specific supplements, such as those for weight loss or performance enhancement, are taken by individuals of a variety of backgrounds ranging from high-performance athletes to sedentary individuals, wishing to improve general health or to treat or ameliorate a specific condition. In younger demographics, particularly athletes, supplements such as creatine are widely used to improve athletic performance (Metzl J D, Small E, Levine S R, Gershel J C. Creatine use among young athletes. Pediatrics. 2001 August; 108(2):421-5). Among older populations dietary supplements are also widely used, often in place of prescription medications (Marinac J S, Buchinger C L, Godfrey L A, Wooten J M, Sun C, Willsie S K. Herbal products and dietary supplements: a survey of use, attitudes, and knowledge among older adults. J Am Osteopath Assoc. 2007 January; 107(1):13-20) orto facilitate dietary requirements which may be lacking.

In the case of certain types of dietary supplements employed for specific goals or benefits, the timing of the administration of the dietary supplement is important; post-workout recovery or pre-workout energetics or intra-workout concentration, for example. It would be advantageous for an individual using such a composition to know approximately when the beneficial effects of dietary supplemental compositions are likely to be manifested post-administration by virtue of being bioavailable. Therefore, it would be advantageous to have, as part of an orally administered dietary supplemental composition, a component which is capable of indicating the bioavailability of the dietary supplement such that the effect of the dietary supplement may be maximized. This may be achieved by a sensory effect perceivable by the individual consuming such a dietary supplement.

### SUMMARY OF THE INVENTION

The foregoing needs and other needs and objectives that will become apparent for the following description are achieved in the present invention, which comprises a substance suitable for administration in conjunction with a dietary supplement, wherein the substance provides transient sensory feedback indicative of the bioavailability of the active components of orally administered dietary supplement compositions.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

A used herein, the term ‘dietary supplement’ includes, nutritional compositions, diet supplements, nutritional supplements, supplemental dietary compositions or those similarly envisioned and termed compositions not belonging to the conventional definition of pharmaceutical interventions as is known in the art. Furthermore, ‘dietary supplements’ as disclosed herein belong to the category of compositions having at least one physiological function when administered to a mammal by conventional routes of administration.

As used herein, the term “sensory feedback substance” refers to any herbal or botanical extract, vitamin, substance, compound, ingredient or molecule capable of inducing, causing or resulting in a sensory effect, sensory feedback, sensory response or sensation in an individual post-administration due to the physiological effects exerted by the herbal or botanical extract, vitamin, substance, compound, ingredient or molecule.

As used herein, the terms “sensory effect”, “sensory feedback”, “sensory response” and “sensation” are used to denote any perceivable effect. The effects may include but are not limited to: flushing, increased or decreased body temperature, perceived increase or decrease in body temperature, sweating, shivering, mild tremors of the extremities, anxiety, an itching or tingling sensation, hyperactivity and hypoactivity. Other sensations may also be perceived by an individual that are suitable for the purposes of the present invention.

The present invention is directed towards the use of specific ingredients as components of orally administered dietary supplement compositions which are capable of providing a safe and transient sensory response perceivable by the user as a means of indicating and estimating the rate and time of bioavailability of the components of the supplemental composition. The user is, via the sensory feedback of the specific ingredient, made aware that their body has begun to absorb the constituents of the dietary supplement. This is particularly advantageous in the case of temporally sensitive administration of substances of dietary supplements. For example, with respect to certain substances, such as proteins, it may be advantageous for the proteins to be bioavailable at certain times in relation to an individual's workout. The addition of a sensory feedback component to a dietary supplement will allow the user to conduct their workout to coincide with the maximal bioavailability of the protein following administration, thereby providing maximal benefits to the user.

Within the scope of the invention, several substances may be used to provide the desired sensory feedback to the user. Furthermore, certain substances will be absorbed faster the others, therefore in another aspect of the present invention, more than one sensory feedback substance may be included to denote additional times following administration of bioavailability beyond the perception of the sensory feedback. For example, if a substance with which it is desirable to supplement the diet of an athlete prior to a workout is provided in a composition with a second substance desirable to supplement the diet of an athlete during a workout, two distinct sensory feedback substances may also be provided in the dietary supplement composition. A first sensory feedback substance being included in the dietary supplement composition provides sensations coinciding with the bioavailability of the first substance and a second sensory feedback substance provides sensations coinciding with the bioavailability of the second substance. In this regard, the user would know the optimum time following administration of the dietary supplement composition to commence a workout.

The sensory feedback substance may also be provided in conjunction with a dietary supplement composition, yet in distinct form not being a component of the dietary supplement form. For example, the dietary supplement may be provided as a drink and the sensory feedback substance being administered to the individual as a pill, capsule or tablet.

In a preferred embodiment of the present invention, Niacin or derivatives of Niacin are included in an orally administered dietary supplement composition. The Niacin or derivatives of Niacin act to provide the user with a perceivable sensation. The sensation is indicative of the bioavailability of the Niacin or derivatives of Niacin and may therefore also be indicative of the bioavailability of additional components of the supplemental composition.

Within the scope of the invention, several other substances may be used to provide the desired sensory feedback to the user. Sensory feedback substances suitable for administration as part of the present invention include, but are not limited to Caffeine anhydrous, Cayenne powder (as Capsicum annuum), Codonopsis pilosula extract, Deanol bitartrate, Evodia rutaecarpa extract (as Tetradium ruticarpum), Guarana powder (as Paullinia cupana), Huperzia serrata extract, Yohimbine HCl, N-acetyl tyrosine, Niacin, Picamilon HCl, Theobroma cacao extract, Thiamin (as thiamine hydrochloride), Thiamin (as thiamine mononitrate), Vinpocetine, Vitamin B12 (as cyanocobalamin), Vitamin B6 (as pyridoxine hydrochloride), Xanthinol nicotinate, Erythroxylum vacciniifolium (small catuaba) extract, Trichilia catigua (big catuaba) extract, Coffea Arabica extract, and Yerba mate (as Ilex paraguariensis). It should be noted, however, that the aforementioned is not an exhaustive list. Other substances which provide a sensory feedback to a user will be apparent to those of skill in the art and are considered by the inventors to be included within the scope of the present invention. Furthermore, it is herein understood by the inventor that the specific dosage respective of the sensory feedback providing substances will vary between the substances and the desired intensity of the sensations. Examples of specific sensory feedback substances are discussed below.

Niacin

Niacin, also known as Vitamin B3 or nicotinic acid is one of several water-soluble B-family vitamins. Niacin is often consumed as a nutritional dietary supplement in the form of a multi-vitamin/mineral complex to improve general health. As a supplement in itself, Niacin has long been successfully used to improve blood lipid profiles (Cheng K, Wu T J, Wu K K, Sturino C, Metters K, Gottesdiener K, Wright S D, Wang Z, O'Neill G, Lai E, Waters M G. Antagonism of the prostaglandin D2 receptor 1 suppresses nicotinic acid-induced vasodilation in mice and humans. Proc Natl Acad Sci USA. 2006 Apr. 25; 103(17):6682-7). In the United States the RDA (Recommended Daily Allowance) for Niacin is 20 mg, while most commercially available multi-vitamin supplements contain at least 25 mg, and some more than 50 mg.

The use of Niacin at pharmacologically active doses is often associated with a flushing effect. This flushing effect may involve a feeling of warmth and an itching sensation due to cutaneous vasodilation of the face, neck and torso that occurs in most individuals (Benyo Z, Gille A, Kero J, Csiky M, Suchankova M C, Nusing R M, Moers A, Pfeffer K, Offermanns S. GPR109A (PUMA-G/HM74A) mediates nicotinic acid-induced flushing. J Clin Invest. 2005 December; 115(12):3634-40). This effect, while negatively affecting compliance among therapeutic users, is considered harmless and may last for about 1 hr (Benyo Z, Gille A, Kero J, Csiky M, Suchankova M C, Nusing R M, Moers A, Pfeffer K, Offermanns S. GPR109A (PUMA-G/HM74A) mediates nicotinic acid-induced flushing. J Clin Invest. 2005 December; 115(12):3634-40). The exact mechanism of the niacin-induced flushing effect is unknown, however, a transient increase in prostaglandin release is thought to be involved (Jungnickel P W, Maloley P A, Vander Tuin E L, Peddicord T E, Campbell J R. Effect of two aspirin pretreatment regimens on niacin-induced cutaneous reactions. J Gen Intern Med. 1997 October; 12(10):591-6).

In various embodiments of the present invention, as detailed in the examples below, Niacin or derivatives of Niacin are in incorporated in a dietary supplemental composition or provided separately to act as an indicator of the bioavailability of various components of the dietary supplemental composition by producing a sensory effect perceivable by the individual consuming said dietary supplemental composition. Preferably, the amount of Niacin or derivatives of Niacin is from about 10 mg to about 100 mg per serving.

Xanthinol Nicotinate

Xanthinol nicotinate is one of several forms of Niacin (vitamin B3). It easily passes through the cell membrane and is considered the most potent form of Niacin. Pharmaceutically, Xanthinol nicotinate is classified as a vasodilator.

In patients with peripheral arterial obliterative disease, Xanthinol nicotinate was found to have anti-platelet and thrombolytic actions accompanied by an increase in the release of NO (Bieron K, Swies J, Kostka-Trabka E, Gryglewski RJ. Thrombolytic and antiplatelet action of xanthinol nicotinate (Sadamin): possible mechanisms. J Physiol Pharmacol. 1998 June; 49(2):241-9). Xanthinol nicotinate may also have the effects of enhancing cellular metabolism and increasing oxygen supply which may be the mechanism of improvements in both short- and long-term memory associated with 500 mg of Xanthinol nicotinate three times per day for eight weeks in a double blind study (Loriaux S M, Deijen J B, Orlebeke J F, De Swart J H. The effects of nicotinic acid and xanthinol nicotinate on human memory in different categories of age. A double blind study. Psychopharmacology (Berl). 1985; 87(4):390-5).

In various embodiments of the present invention, as detailed in the examples below, Xanthinol nicotinate or derivatives of Xanthinol nicotinate are in incorporated in a dietary supplemental composition or provided separately to act as an indicator of the bioavailability of various components of the dietary supplemental composition by producing a sensory effect perceivable by the individual consuming said dietary supplemental composition. Preferably, the amount of Xanthinol nicotinate or derivatives of Xanthinol nicotinate is from about 10 mg to about 500 mg per serving.

Picamilon

Picamilon is the sodium salt of N-nicotinoyl-gamma-aminobutyric acid and is generally referred to a specific binding of niacin and γ-aminobutyric acid (GABA). Due to containing both niacin and GABA, Picamilon is expected to confer perceivable sensory effects. Picamilon has been widely used in Russia for the treatment of a number of conditions including depression, anxiety, headache, and brain injury.

In various embodiments of the present invention, as detailed in the examples below, Picamilon or derivatives of Picamilon are in incorporated in a dietary supplemental composition or provided separately to act as an indicator of the bioavailability of various components of the dietary supplemental composition by producing a sensory effect perceivable by the individual consuming said dietary supplemental composition. Preferably, the amount of Picamilon or derivatives of Picamilon is from about 10 mg to about 500 mg per serving.

Yohimbine

Yohimbine is a naturally occurring alkaloid derived from the bark of the African tree, Pausinysatlia yohimbe. Traditionally, yohimbine has been used as an aphrodisiac in traditional medicine and additionally as a treatment for erectile dysfunction. The main identified biological activity of yohimbine is as an alpha(2) adrenergic blocker or antagonist (Cameron O G, Zubieta J K, Grunhaus L, Minoshima S. Effects of yohimbine on cerebral blood flow, symptoms, and physiological functions in humans. Psychosom Med. 2000 July-August; 62(4):549-59). Blockade of alpha(2) adrenoreceptors by yohimbine has the effect of stimulating the sympathetic nervous system (Le Corre P, Parmer R J, Kailasam M T, Kennedy B P, Skaar T P, Ho H, Leverge R, Smith D W, Ziegler M G, Insel P A, Schork N J, Flockhart D A, O'connor DT. Human sympathetic activation by alpha2-adrenergic blockade with yohimbine: Bimodal, epistatic influence of cytochrome P450-mediated drug metabolism. Clin Pharmacol Ther. 2004 August; 76(2):139-53), which is expected to produce perceivable sensory effects.

In various embodiments of the present invention, as detailed in the examples below, Yohimbine or derivatives of Yohimbine are in incorporated in a dietary supplemental composition or provided separately to act as an indicator of the bioavailability of various components of the dietary supplemental composition by producing a sensory effect perceivable by the individual consuming said dietary supplemental composition. Preferably, the amount of Yohimbine or derivatives of Yohimbine is from about 1 mg to about 10 mg per serving.

Trichilia catigua

The Trichilia catigua tree is native to Brazil where the bark from the tree has been used in traditional medicines as central nervous system stimulant. In vitro and in vivo studies in mice and rats have shown that Trichilia catigua extracts have anti-depressant effects which are likely mediated by increased release and decreased uptake of the neurotransmitters serotonin and dopamine (Campos M M, Fernandes E S, Ferreira J, Santos A R, Calixto J B. Antidepressant-like effects of Trichilia catigua (Catuaba) extract: evidence for dopaminergic-mediated mechanisms. Psychopharmacology (Berl). 2005 October; 182(1):45-53).

In various embodiments of the present invention, as detailed in the examples below, Trichilia catigua or derivatives of Trichilia catigua are in incorporated in a dietary supplemental composition or provided separately to act as an indicator of the bioavailability of various components of the dietary supplemental composition by producing a sensory effect perceivable by the individual consuming said dietary supplemental composition. Preferably, the amount of Trichilia catigua or derivatives of Trichilia catigua is from about 10 mg to about 500 mg per serving.

In a preferred embodiment of the present invention, Niacin or a derivative of Niacin is incorporated as an active ingredient in a dietary supplemental composition or provided separately. The Niacin or derivative of Niacin act to provide the individual having consumed the dietary supplemental composition with a safe, transient, perceivable sensation indicative of the bioavailability of the Niacin or derivative of Niacin which will further indicate the bioavailability of other active ingredients in the dietary supplemental composition. The amount of Niacin or derivative of Niacin provided via administration to an individual should be of an amount sufficient to induce the sensory flushing effect. The preferred the preferred derivative of Niacin is Xanthinol nicotinate.

Not wishing to be bound by theory, it is believed that any perceivable sensation resulting from oral ingestion of one or more sensory feedback substances, which are components of a multi-component dietary supplemental composition, is indicative of the bioavailability of the dietary supplement as a whole. It is understood by the inventor that the various active components of a multi-component dietary supplemental composition may likely have differing rates of bioavailability. These differing rates of bioavailability may be due to a number of variables including but not limited to: chemical structure, mechanism of uptake, post-uptake metabolism and specific site of biological activity. It is however believed by the inventors that the rate of bioavailability of an indicator such Niacin or derivatives of Niacin or of Xanthinol nicotinate or a derivative of Xanthinol nicotinate will be indicative of the average rate or earliest rate of bioavailability of most of the active components of a dietary supplemental composition. It is also understood by the inventor that Niacin or derivatives of Niacin or Xanthinol nicotinate or a derivatives of Xanthinol nicotinate or other sensory feedback substances can be used by the individual to gauge the time of bioavailability of other components of dietary supplement composition.

In addition to providing the user of the dietary supplemental composition containing an indicator of bioavailability with an indication for the purposes of timing a workout or the bioavailability of other substance included therein, it may also advantageously provide the user with a perception of usefulness or effectiveness. Particularly when consumed as part of a pre-workout dietary supplemental composition, the sensory effects of sensory feedback substances may act to enhance or accentuate the normal effects of moderate to intense physical activity. As a further benefit, Niacin or derivatives of Niacin or Xanthinol nicotinate or derivatives of Xanthinol nicotinate may provide the user with the health benefits previously disclosed above and normally attributed to Niacin as a stand-alone vitamin supplement.

In another aspect of the present invention, the perceptible sensory effects due to sensory feedback substances are useful as an indicator of the bioavailability for the purpose of formulating various controlled-release formats. Such controlled-release formats, as are commonly known in the art, include timed- or time-release, sustained-release and delayed-release. Through this aspect, the effectiveness of a controlled-release composition may be readily tested in a biologically relevant in vivo system.

Additional embodiments of the present invention may also include portions of the composition as nano-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference, discloses a method of achieving a solid oral dosage form with multiple dissolution characteristics for the release of active ingredients.

Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent.

According to various embodiments of the present invention, the dietary supplemental compositions into which sensory feedback substances are incorporated may be consumed in any form. For instance, the dosage form of the dietary supplement may be provided as, e.g., a powder beverage mix, a liquid beverage, a ready-to-eat bar or drink product, a capsule, a liquid capsule, a tablet, a caplet, or as a dietary gel. The sensory feedback substance may also be administered separately to an individual as a tablet, pill, capsule, caplet, or the like concomitantly with the dietary supplement or within a finite period before or after administration of the dietary supplement so as to act as gauge for the user as to the bioavailability of a dietary supplement composition.

Furthermore, the dosage form of the dietary supplement may be provided in accordance with customary processing techniques for herbal and nutritional supplements in any of the forms mentioned above. Also, the dosage form of a separately administered sensory feedback substance may be provided in accordance with customary processing techniques for herbal and nutritional supplements in any of the forms mentioned above. Additionally, the dietary supplement set forth in the example embodiments herein may contain any appropriate number and type of excipients, as is well known in the art.

Although the following examples illustrate the practice of the present invention in several of its embodiments, the examples should not be construed as limiting the scope of the invention. Other embodiments will be apparent to one of skill in the art from consideration of the specification and examples.

### EXAMPLES

The examples herein described are provided to ensure a thorough understanding of the invention and are not intended to be limiting. Those of skill in the art will readily appreciate additional embodiments within the scope of the present invention.

### Example 1

### Testing the Efficacy of Niacin in Capsule Form

In order to test the ability of orally administered Niacin to induce a sensory effect a test was conducted. Five subjects were given a capsule containing Niacin and asked to report on any perceived sensory effects. Each capsule contained 30 mg of Niacin and filled to 150 mg with lactose as a filler-binder. Subjects were informed of the capsule contents and of possible effects. Subjects were asked to report any sensory effects felt, the time to onset and the duration of any effects. Table 1 shows the results of a single 30 mg dose of oral Niacin.

TABLE 1
Effects of Orally Administered Niacin

| | Sensory | | | |
|---|---|---|---|---|
| Subject | Effect(s) | Time to Onset | | Duration |
| 1 | tingling on | 20 | min | 15 min |
| | face/head | | | |
| 2 | warm feeling | 15 | min | 45 min |
| | over whole body | | | |
| 3 | none | — | | — |
| 4 | warm feeling | 10-15 | min | 45 min |
| | over whole body | | | |

| | | | |
|---|---|---|---|
| 5 | none | — | — |

As can be noted from Table 1, three of the five subjects experienced transient sensory effects after administration of Niacin. Thus, in the case of the data presented in Table 1, the Niacin can be consumed by an individual either as a component of a dietary supplement or separately in conjunction with a dietary supplement. The individual perceives a sensory effect within approximately 15 minutes. In a case where the dietary supplement, for example, is a pre-workout energy supplement the individual, by way of the perceived sensory effect, is prompted to begin an exercise session approximately 15 minutes after consuming the dietary supplement. Wherein the Niacin is a component of a dietary supplement the individual uses the perceived sensory effects to guide the commencement of all exercise sessions for which the dietary supplement is to be consumed. In cases where the Niacin is taken as a separate component in conjunction with the dietary supplement, the perceived sensory effects from one or more administrations of Niacin is used as a guide for the timing of subsequent dietary supplement administrations in relation to exercise commencement. The sensory effects reported by individuals having taken Niacin lasted 15 to 45 minutes and would thus dissipate throughout the course of a typical exercise session.

### Example 2

### Testing the Efficacy of Picamilon in Capsule Form

In order to test the ability of orally administered Picamilon to induce a sensory effect a test was conducted. Six subjects were given a capsule containing Picamilon and asked to report on any perceived sensory effects. Each capsule contained 100 mg of Picamilon and filled to 150 mg with lactose as a filler-binder. Subjects were informed of the capsule contents and of possible effects. Subjects were asked to report any sensory effects felt, the time to onset and the duration of any effects. Table 2 shows the results of a single 100 mg dose of oral Picamilon.

TABLE 2
Effects of Orally Administered Picamilon

| | Sensory | | | | |
|---|---|---|---|---|---|
| Subject | Effect(s) | Time to Onset | | Duration | |
| 1 | warmth and | 10 min (warmth) | | 5 min (warmth) | |
| | tingling - head | 30 min | | 15 min | |
| | and arms | (tingling) | | (tingling) | |
| 2 | none | — | | — | |
| 3 | tingling | 15 | min | 15-20 | min |
| 4 | none | — | | — | |
| | increased | | | | |
| | feeling of | | | | |
| | alertness and | | | | |
| 5 | relaxation | 15-20 | min | 1.5 | hr |
| 6 | warmth - body | 15-20 | min | 45 | min |

As can be noted from Table 2, four of the six subjects reported noticeable effects after administration of Picamilon. Picamilon is a derivative of Niacin formed by reacting Niacin with the neurotransmitter Gamma-aminobutyric acid (GABA) and, as such, is expected to have effects similar to Niacin. As with Niacin, discussed above, the administration of Picamilon, via perceived sensory effect, is used guide the timing of administration of a dietary supplement in relation to the intended use of the dietary supplement. The perceived sensory effects of Picamilon, their onset time and duration were similar to those of Niacin and may thus be employed in a similar fashion.

### Example 3

### Testing the Efficacy of Yohimbine in Capsule Form

In order to test the ability of orally administered Yohimbine to induce a sensory effect a test was conducted. Four subjects were given a capsule containing Yohimbine and asked to report on any perceived sensory effects. Each capsule contained 5 mg of Yohimbine and filled to 150 mg with lactose as a filler-binder. Subjects were informed of the capsule contents and of possible effects. Subjects were asked to report any sensory effects felt, the time to onset and the duration of any effects. Table 3 shows the results of a single 5 mg dose of oral Yohimbine.

TABLE 3
Effects of Orally Administered Yohimbine

| | Sensory | | | | |
|---|---|---|---|---|---|
| Subject | Effect(s) | Time to Onset | | Duration | |
| 1 | increased | 30 | min | 1 | hr |
| | energy and | | | | |
| | focus | | | | |
| 2 | increased | 15 | min | 2 | hr |
| | energy and | | | | |
| | redness | | | | |
| 3 | warmth and | 1 | hr | 10-15 | min |
| | tingling (head) | | | | |

| | | | |
|---|---|---|---|
| 4 | none | — | — |

As can be noted from Table 3, three of the four subjects reported noticeable effects after administration of Yohimbine. As in the case with Niacin and Picamilon presented above, the administration of Yohimbine results in perceivable sensory effects. On average however, the perceived sensory effects from Yohimbine were characterized by both a longer onset time and longer duration than Niacin and Picamilon. It is used in supplements wherein the desired time to sensory perception is longer than that of Niacin or Picamilon. Yohimbine also differed in the general type of perceived sensory effects, having a higher propensity to induce perceptions of increased energy and focus. Therefore, Yohimbine may be employed in situations different from that of Niacin or Picamilon. For example, many weight loss supplements are to be consumed 30 to 60 minutes before a meal; the sensory effect profile of Yohimbine is suitable for indicating the time post-administration of a weight loss supplement to commence eating a meal. Yohimbine may also be useful in a pre-workout energy supplement intended to be consumed at a time earlier than immediately before an exercise session; such dietary supplements may include proteins, which may require a period of time for digestion before exercise is commenced.

### Example 4

### Testing the Efficacy of Trichilia catigua in Capsule Form

In order to test the ability of orally administered Trichilia catigua to induce a sensory effect a test was conducted. Eight subjects were given two capsules containing Trichilia catigua before exercise and asked to report on any perceived sensory effects. Each capsule contained 50 mg of Trichilia catigua and filled to 150 mg with lactose as a filler-binder. Subjects were informed of the capsule contents and of possible effects. Subjects were asked to report any sensory effects felt, the time to onset and the duration of any effects. Table 4 shows the results of a single 100 mg dose of oral Trichilia catigua.

TABLE 4
Effects of Orally Administered Trichilia catigua

| Subject | Sensory Effect(s) | Time to Onset | | Duration | |
|---|---|---|---|---|---|
| 1 | none | — | | — | |
| 2 | Increased energy, | 30-40 | min | 30 | min |
| | drive and | | | | |
| | sweating | | | | |
| 3 | none | — | | — | |
| 4 | Increased energy, | 30 | min | 120 | min |
| | focus, vitality, | | | | |
| | drive | | | | |
| 5 | Increased energy, | 15 | min | 90 | min |
| | alertness, | | | | |
| | sweating | | | | |
| 6 | none | — | | — | |
| 7 | Increased energy, | 20 | min | 30-40 | min |
| | alertness | | | | |
| 8 | Increased energy, | 30 | min | 60-90 | min |
| | focus | | | | |

As can be noted from Table 4, five of the eight subjects experienced transient sensory effects after administration of Trichilia catigua. Thus, in the case of the data presented in Table 4, the Trichilia catigua can be consumed by an individual either as a component of a dietary supplement or separately in conjunction with a dietary supplement. The individual perceives a sensory effect within approximately 15 to 30 minutes. In a case where the dietary supplement, for example, is a pre-workout energy supplement the individual, by way of the perceived sensory effect, is prompted to begin an exercise session approximately 30 minutes after consuming the dietary supplement. Wherein the Trichilia catigua is a component of a dietary supplement the individual uses the perceived sensory effects to guide the commencement of all exercise sessions for which the dietary supplement is to be consumed. In cases where the Trichilia catigua is taken as a separate component in conjunction with the dietary supplement, the perceived sensory effects from one or more administrations of Trichilia catigua is used as a guide for the timing of subsequent dietary supplement administrations in relation to exercise commencement. The sensory effects reported by individuals having taken Trichilia catigua lasted 30 to 120 minutes and would thus dissipate throughout the course of a typical exercise session.

The results of Examples 1-4 indicate that it is possible to control both the time of onset and duration of effects, as well as the type of effects, through a combination of specific amounts of Niacin, Picamilon, Yohimbine and Trichilia catigua. Therefore, one may envision a sensory feedback profile characterized by two or more different onset times, each differing in duration and possibly type of sensory effect, to aid in informing the user of the bioavailability of more than one component of a dietary supplement. The dietary supplement may incorporate the multiple sensory feedback substances or be administered in conjunction with said dietary supplement. When used in temporal relation to a dietary supplement, such a sensory feedback profile may be employed to inform the individual, via bioavailability, of the optimal time for a number of activities such as exercise commencement time, exercise cessation time or meal time.

### Example 5

### Incorporating Sensory Effects into a Multi-Ingredient Composition for Weight Control

The inventor further examined the efficacy of adding a sensory effect to a multi-ingredient dietary supplemental composition. A Weight Control composition was chosen and is shown in Table 5.

TABLE 5
Weight Control supplement composition.

| Ingredient | Amount (g) |
|---|---|
| Caffeine Anhydrous | 0.2000 |
| Cissus quadrangularis extract (from stem | 0.1500 |
| and leaves) | |
| Green Tea dry leaf extract (Camellia | 0.1222 |
| sinensis) | |
| Soy Albumin bean extract (Glycine max) | 0.0500 |
| Vitamin B-6 (pyridoxine hydrochloride) | 0.0500 |
| L-Selenomethionine | 0.0120 |
| Cyanocobalamin | 0.0050 |
| Chromium Polynicotinate | 0.0015 |
| Folic Acid | 0.0004 |

To the Weight Control Supplement of Table 5 were added 50 mg of Niacin and a further 50 mg of Caffeine Anhydrous in separate capsule. The composition was given to eighteen subjects (5 male, 13 female) without disclosing the added ingredients. Subjects were given two servings of the supplement and asked to consume one serving and report and physiological changes noticed within 30 to 45 minutes after administration. Each serving consisted of two capsules of the Weight Control Supplement and one capsule of containing the Niacin and Caffeine blend to provide a sensory effect to the subjects. Administration of the second serving was optional two to three hours after the first serving. A summary of the results is presented in Table 6.

TABLE 6
Results of adding sensory effect to a weight control supplement.

| Subject | Sex | Tingling | Warmth | Flushing |
|---|---|---|---|---|
| 1 | M | | | |
| 2 | M | ✓ | | ✓ |
| 3 | M | ✓ | | |
| 4 | M | ✓ | | |
| 5 | M | | | |
| 6 | F | ✓ | | ✓ |
| 7 | F | | | |
| 8 | F | | | |
| 9 | F | ✓ | ✓ | ✓ |
| 10 | F | ✓ | | |
| 11 | F | ✓ | ✓ | ✓ |
| 12 | F | ✓ | ✓ | |
| 13 | F | | | |
| 14 | F | | | |
| 15 | F | ✓ | | |
| 16 | F | | | |
| 17 | F | | | ✓ |
| 18 | F | | | |

As can be noted from Table 6, ten out of the eighteen subjects experienced sensory effects consistent with the expected effects of Niacin. The weight control supplement is intended to be consumed before meals. Therefore, the inclusion of ingredients to cause transient sensory effects may serve as a useful indicator of an optimal time, post-administration of the supplement, at which to consume a meal so as to gain the intended benefit i.e. in the case of this specific example, weight loss or reduced weight gain from the Weight Control Supplement.

### Example 6

### A Possible Pre-Workout Supplement

A dietary supplement to provide sustained energy for intense workouts is provided in the form of a capsule to be consumed with water before exercise. A serving of the dietary supplement contains the following:
about 0.500 g of Green tea dry leaf extract, about 0.300 g of Caffeine anhydrous, about 0.035 g of Niacin, about 0.100 g of Xanthinol nicotinate, about 0.100 g of N-Acetyl-L-Tyrosine and about 0.001 g of Guarana powder from seed.
   The perceptible sensory effects due to the incorporated Niacin and Xanthinol nicotinate provides the user with the optimal timing for conducting an intense exercise session post-administration of the pre-workout energy supplement and further provide a feeling of efficacy and energy.

### Example 7

### Testing a Delayed-Release Tablet Formulation

The efficacy of a tablet designed to delay the release of active components for approximately 1 hour post-administration may be tested by comparison to a conventional immediate-release tablet as outlined below using the sensory feedback mechanism of the present invention.

Samples: Tablets are formulated into four types—immediate release Niacin (50 mg), delayed release Niacin (50 mg), immediate release placebo and delayed release placebo.
Study Design Subjects are asked to refrain from taking supplements containing Niacin or derivatives thereof. Subjects are randomly assigned to one of four groups. Tablets are administered to subjects in the morning on an empty stomach and monitored over the course of several hours for the time of onset of a Niacin flushing effect. One-week later, the subjects are randomly re-assigned and the experiment is repeated. In this regard, the present invention may be employed to aid in the development of time-dependant release compositions. Specifically, the time-release mechanism can be safely tested in vivo and an observer can record results independent of the subject. Therefore providing a more objective method of testing in vivo time-release composition platforms

### EXTENSIONS AND ALTERNATIVES

In the foregoing specification, the invention has been described with specific embodiments thereof; however, it will be evident that various modifications and changes may be made thereto without departing from the broader spirit and scope of the invention.

What is claimed:

1. A method for determining post-ingestion bioavailability of a dietary supplement by an individual comprising the co-administration to the individual a first substance which provides transient sensory feedback to the individual and at least a second substance;
whereby the bioavailability of the second substance is indicated to the individual via transient sensory feedback to the individual derived from the first substance.

2. The method claim 1 wherein the second substance is a dietary supplement.

3. The method of claim 2 wherein the transient sensory feedback provided to the individual substantially coincides with the optimal bioavailability of at least one substance included in the dietary supplement.

4. The method of claim 3 wherein the substance which provides a transient sensory feedback to the individual post-ingestion indicates an optimal time for the individual to commence a desired activity.

5. The method of claim 4 wherein the desired activity is selected from the group consisting of exercise and food consumption.

6. The method of claim 1 wherein the sensory feedback substance is fine-milled.

7. The method of claim 1 wherein the sensory feedback substance is provided in a controlled-release format.

8. The method of claim 1 wherein the sensory feedback substance is selected from the group consisting of Niacin or derivatives of Niacin, Xanthinol nicotinate or a derivatives of Xanthinol nicotinate, Caffeine or a derivatives of Caffeine, Cayenne powder, Codonopsis pilosula extract, Deanol bitartrate, an extract of Evodia rutaecarpa, Guarana powder, an extract of Huperzia serrata, Yohimbine or derivatives of Yohimbine, an extract of Yohimbe, N-acetyl tyrosine, Picamilon, an extract of Theobroma cacao, Thiamin or a derivatives of Thiamine, Cyanocobalamin, Pyridoxine, Vinpocetine, Yerba mate, Coffea arabica extract, Erythroxylum vacciniifolium (small catuaba) extract and Trichilia catigua (big catuaba) extract.

9. A transient sensory feedback method for informing a user of the bioavailability of an orally administered composition, the composition comprising at least a sensory feedback substance and a dietary supplement.

10. The method of claim 9 wherein the transient sensory feedback substantially coincides with the optimal bioavailability of at least one substance included in the dietary supplement.

11. The method of claim 10 wherein the substance which provides the transient sensory feedback to the individual post-ingestion indicates an optimal time for the individual to commence a desired activity.

12. The method of claim 11 wherein the desired activity is selected from the group consisting of exercise and food consumption.

13. The method of claim 9 wherein the sensory feedback substance is fine-milled.

14. The method of claim 9 wherein the sensory feedback substance is in a controlled-release format.

15. The method of claim 9 wherein the sensory feedback substance is selected from the group consisting of Niacin or derivatives of Niacin, Xanthinol nicotinate or a derivatives of Xanthinol nicotinate, Caffeine or a derivatives of Caffeine, Cayenne powder, Codonopsis pilosula extract, Deanol bitartrate, an extract of Evodia rutaecarpa, Guarana powder, an extract of Huperzia serrata, Yohimbine or derivatives of Yohimbine, an extract of Yohimbe, N-acetyl tyrosine, Picamilon, an extract of Theobroma cacao, Thiamin or a derivatives of Thiamine, Cyanocobalamin, Pyridoxine, Vinpocetine, Yerba mate, Coffea arabica extract, Erythroxylum vacciniifolium (small catuaba) extract and Trichilia catigua (big catuaba) extract.
